(19) United States
(12) Patent Application Publication
(10) Pub. No.: US 2023/0001071 A1
(43) Pub. Date: Jan. 5, 2023

(54) PUMP WITH ELECTRONICS MOUNTED IN A SUSPENDED MANNER

(71) Applicant: B. Braun Melsungen AG, Melsungen (DE)

(72) Inventors: Jan Schwarz, Melsungen (DE); Christoph Erlen, Kassel (DE); Hans-Josef Gerlach, Marsberg (DE); Joachim Schuetz, Fulda (DE); Gerhard Schoeffel, Blaustein (DE); Daniel Wolf, Bibertal (DE)

(21) Appl. No.: 17/849,818

(22) Filed: Jun. 27, 2022

(30) Foreign Application Priority Data
Jul. 1, 2021 (DE) 20 2021 103 518.8

(51) Int. Cl. A61M 5/142

(52) U.S. Cl. CPC A61M 5/142; A61M 2205/82; A61M 2205/215; A61M 2205/50

(57) ABSTRACT

A medical fluid pump with an upper housing shell, a lower housing shell and a front lid pivotably hinged to the lower housing shell. A printed circuit board with essential control devices is arranged in the upper housing shell, and an energy storage is provided and fixed in the lower housing shell.

### CROSS-REFERENCE TO RELATED APPLICATION

This application claims priority under 35 U.S.C. § 119 to German Application No. 20 2021 103 518.8, filed Jul. 1, 2021, the content of which is incorporated by reference in its entirety.

### FIELD

The disclosure relates to a medical fluid pump with electronics mounted in a suspended manner.

### BACKGROUND

In medicine, fluid pumps, in particular syringe pumps and peristaltic pumps, are widely used to supply a patient with a defined dose of medication. The fluid pump contains various electronic components such as a drive, a control device and a power adapter. Since the fluid pump is provided and formed to administer a fluid, there is a risk of fluid leakage. The leaking fluid can cause short circuits in the electronic components of the fluid pump and thus damage or destroy the electronic components.

If this occurs during medication administration, for example, it may have serious consequences for the patient.

### SUMMARY

The object of the present invention is therefore to increase the safety of medical pumps and, in particular, to arrange electronic components critical for treatment in a fluid pump in such a way that damage to the electrical components is prevented as far as possible even in the event of entry of fluid.

This object is solved by the medical fluid pump with electronics mounted in a suspended manner according to the invention. In this context, suspended means the absence of additional support members or bearings.

The medical fluid pump, in particular a syringe pump or a peristaltic pump, includes an upper housing shell, a lower housing shell, and a front lid pivotably hinged to the lower housing shell, wherein a printed circuit board with the essential control devices is fixed/arranged/mounted in the upper housing shell, in particular in a suspended manner, and an energy storage is provided and fixed in the lower housing shell.

In other words, the fluid pump has a housing that includes the upper housing shell and the lower housing shell. The front lid is hinged to the lower housing shell, which preferably contains operating elements and/or a display device. In the upper housing shell, the printed circuit board with the essential control devices is fixed, wherein the printed circuit board is preferably fixed/arranged suspended at a distance from the upper housing shell. In the lower housing shell, the energy storage device is provided and fixed, which is electrically connected to the printed circuit board.

Attaching the printed circuit board to the upper housing shell ensures that, in the event of fluid entering the fluid pump, the printed circuit board does not come into contact with the fluid that accumulates in the lower housing shell. This prevents the fluid from short-circuiting the printed circuit board or damaging the printed circuit board through corrosion. The suspended attachment to the upper shell also ensures that the fluid does not migrate to the printed circuit board via capillary forces. The energy storage itself is insensitive to fluids and the critical point of connection of the energy storage to the printed circuit board is at the printed circuit board.

In a first preferred aspect, a drive of the medical fluid pump is fixed/arranged/mounted in the upper housing shell, in particular in a suspended manner.

In other words, the drive, which includes, for example, a motor, a transmission, and a drive spindle, is fixed to the upper housing shell in a force-fit and/or form-fit manner.

Attaching the drive to the upper housing shell ensures that, in the event of fluid entering the fluid pump, the drive does not come into contact with the fluid that accumulates in the lower housing shell. In addition to preventing short circuits and corrosion, it can also prevent lubricants applied to the motor, transmission and/or drive spindle for friction reduction from being washed off by the fluid. Washing off of the lubricants results in increased wear in the drive.

In a further preferred aspect, a power adapter is fixed/arranged/mounted in the upper housing shell, in particular in a suspended manner.

In other words, a power adapter, which preferably contains a transformer and supplies the electrical components of the fluid pump with electrical current, is fixed to the upper housing shell in a force-fit and/or form-fit manner. The power adapter is preferably operated with a mains voltage.

Attaching the power adapter to the upper housing shell ensures that, in the event of fluid entering the fluid pump, the power adapter does not come into contact with the fluid that accumulates in the lower housing shell. This prevents short circuits in the power adapter and, since the power adapter is operated with mains voltage, also reduces the danger to operators.

In a further preferred aspect, a communication module is fixed/arranged/mounted in/on the upper housing shell, in particular in a suspended manner.

In other words, in the medical fluid pump, the communication module is fixed in/on the upper housing shell in a force-fit and/or form-fit manner.

Attaching the communication module to the upper housing shell ensures that, in the event of fluid entering the fluid pump, the communication module does not come into contact with the fluid that accumulates in the lower housing shell.

In a further preferred aspect, the drive is arranged between the printed circuit board and the lower housing shell.

In other words, the drive is attached to the upper housing shell such that the printed circuit board is arranged between the drive and the upper housing shell. Preferably, the drive is fixed to the upper housing shell via drive support members arranged through through holes of the printed circuit board.

In a further preferred aspect, the printed circuit board is provided and fixed/arranged/mounted on suspension devices spaced from the upper housing shell.

In other words, the printed circuit board does not lie directly against the upper housing shell, but is fixed in a force-fit and/or form-fit manner at a distance from the upper housing shell via suspension devices/spacers. Preferably, the spacers are columnar receptacles formed in one piece with the upper housing shell, to which the printed circuit board is screwed or glued or welded or riveted.

The distance of the printed circuit board from the upper housing shell ensures that air can flow around the printed circuit board. This prevents condensation from forming between the printed circuit board and the upper housing shell. In addition, the air flowing around the printed circuit board cools the printed circuit board and prevents the printed circuit board from overheating.

In a further preferred aspect, a linear potentiometer is provided and fixed/arranged/mounted in/on the lower housing shell.

In other words, in the medical fluid pump in the form of the syringe pump, the linear potentiometer is fixed in the lower housing shell in a force-fit and/or form-fit manner.

In a further preferred aspect, the power adapter is fixed/arranged/mounted to a portion of the upper housing shell that is normal/perpendicular to the printed circuit board.

In other words, the power adapter is arranged on a rear side of the housing, which faces the front lid and is formed by the upper housing shell.

Attaching the power adapter perpendicular to the printed circuit board ensures that sufficient air flows around both components and that both components are cooled sufficiently. In addition, attaching the power adapter to the rear side of the housing allows a heat sink to be provided.

### BRIEF DESCRIPTION OF THE DRAWING FIGURES

FIG. 1 is an illustration of a medical fluid pump according to the invention in the form of a syringe pump.

FIG. 2 is a perspective view of an inside of an upper housing shell of a housing of the medical fluid pump and a printed circuit board.

FIG. 3 is an illustration of the inside of the upper housing shell of the medical fluid pump and the printed circuit board, a drive and a power adapter.

### DETAILED DESCRIPTION

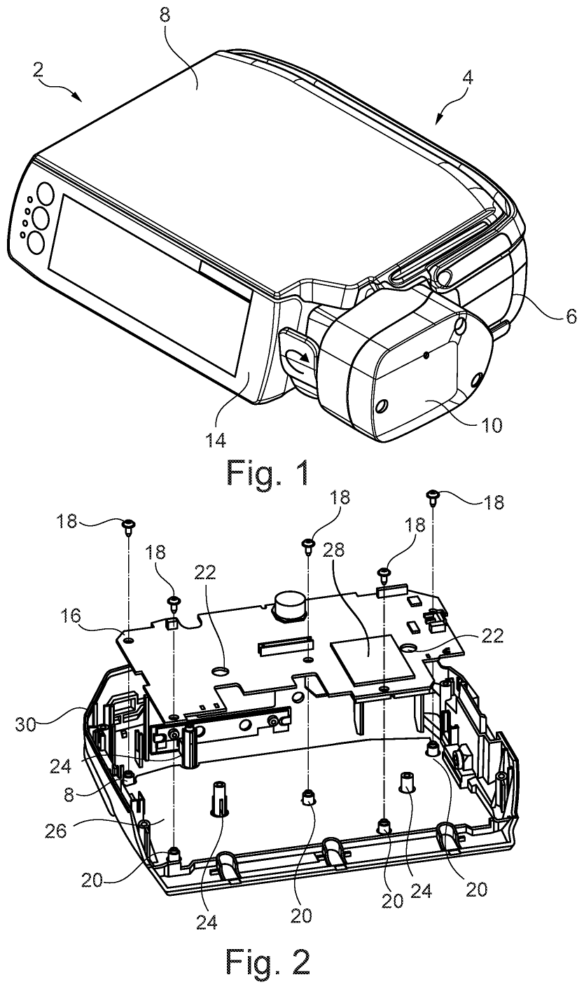

FIG. 1 shows a medical fluid pump according to the invention, in particular a syringe pump 2. The basic mode of operation of the syringe pump 2 is sufficiently described in EP0566825A1, so that a detailed description of the syringe pump 2 is omitted here.

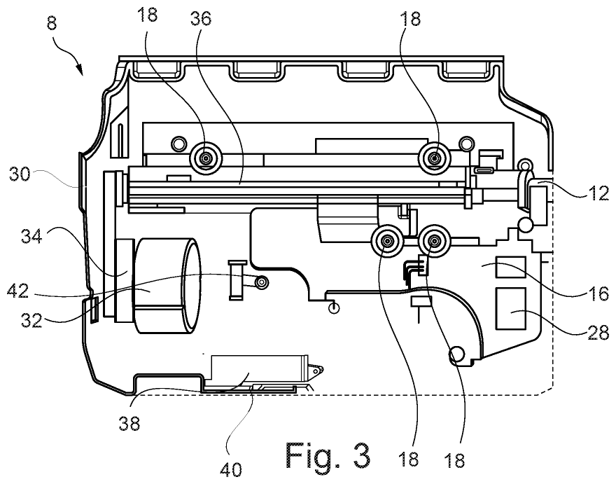

The syringe pump 2 shown in FIG. 1 includes a housing 4 with a lower housing shell 6 and an upper housing shell 8. A drive head 10 is connected to the syringe pump 2 via a holding arm 12 (see FIG. 3) so that it can move relative to the housing 4. A front lid 14 is provided and formed on the front side of the syringe pump 2. The front lid 14 is foldably hinged to the lower housing shell 6 via hinges. The syringe pump 2 is an exemplary example of a medical fluid pump. The present invention is also applicable to other medical fluid pumps, such as a peristaltic pump.

FIG. 2 shows a perspective view of the upper housing shell 8 of the housing 4 of the medical fluid pump according to the invention. In a mounted state, the upper housing shell 8 is in contact with the lower housing shell 6 via the contact rim 30. A printed circuit board 16 is provided and formed to be fixed via screws 18 to support members 20, which are preferably formed in one piece with the upper housing shell 8. The printed circuit board 16 has through holes 22, each of which is penetrated by a drive support member 24, which is provided and formed to have a drive fixed thereto in a force-fit and/or form-fit manner. A height of the drive support member 24 is greater than a height of the support member 20, each with respect to an inner surface 26 of the upper housing shell 8. The printed circuit board 16 includes a control device 28 of the medical fluid pump. In a mounted state, the printed circuit board 16 is spaced apart from the contact rim 30 in a direction normal to the printed circuit board. In other words, the printed circuit board 16 is spaced from the lower housing shell 6 in a mounted state.

FIG. 3 shows the upper housing shell 8 of the housing of the medical fluid pump according to the invention in a top view. In FIG. 3, the upper housing shell 8 is shown in a state in which the drive, consisting of a motor 32, a transmission 34 and a drive spindle 36 are fixed in the upper housing shell 8. In addition, a power adapter 38 is fixed to the upper housing shell 8. The power adapter 38 is preferably fixed to a portion 40 of the upper housing shell 8, which is oriented perpendicular to the printed circuit board 16. The motor 32, transmission 34, drive spindle 36, and power adapter 38 are preferably fixed in the upper housing shell 8 via screws 18. The screws 18 are preferably screwed into the drive support members 24. In addition to the drive support member 24 and the support member 20, further support member elements may be provided and formed to fix individual components or assemblies to the upper housing shell 8 at a distance from the lower housing shell 6 and preferably at a distance from the upper housing shell 8. Additionally, a communication module 42 is attached to the upper housing shell 8. The communication module 42 is preferably a wireless communication module, but may also be formed as a wired communication module that communicates information and data processed in the control device 28 located on the printed circuit board 16.

1. A medical fluid pump comprising:
an upper housing shell;
a lower housing shell;
a front lid pivotably hinged to the lower housing shell;
a printed circuit board with essential control devices; and
an energy storage,
the printed circuit board being arranged in the upper housing shell, and
the energy storage being provided and fixed in the lower housing shell.

2. The medical fluid pump according to claim 1, further comprising a drive arranged in the upper housing shell.

3. The medical fluid pump according to claim 2, wherein the drive is arranged between the printed circuit board and the lower housing shell.

4. The medical fluid pump according to claim 2, wherein the drive is arranged in the upper housing shell in a suspended manner.

5. The medical fluid pump according to claim 1, further comprising a power adapter arranged in the upper housing shell.

6. The medical fluid pump according to claim 5, wherein the power adapter is fixed to a portion of the upper housing shell that is perpendicular to the printed circuit board.

7. The medical fluid pump according to claim 1, further comprising a communication module arranged in the upper housing shell.

8. The medical fluid pump according to claim 1, wherein the printed circuit board is provided and arranged on spacers spaced from the upper housing shell.

9. The medical fluid pump according to claim 1, further comprising a linear potentiometer arranged in the lower housing shell.

10. The medical fluid pump according to claim 1, wherein the printed circuit board is arranged in the upper housing shell in a suspended manner.
